(19) United States
(12) Patent Application Publication
(10) Pub. No.: US 2023/0157583 A1
(43) Pub. Date: May 25, 2023

(54) SYSTEM, METHOD, AND APPARATUS FOR SEXUAL PERFORMANCE SENSING

(71) Applicant: ALEXANDER WARNER, TORONTO (CA)

(72) Inventor: ALEXANDER WARNER, TORONTO (CA)

(21) Appl. No.: 17/534,733

(22) Filed: Nov. 24, 2021

(51) Int. Cl. A61B 5/11; A61B 5/00

(52) U.S. Cl. CPC A61B 5/1123; A61B 5/742; A61B 5/0004; A61H 19/44

(57) ABSTRACT

A sexual performance sensing apparatus includes a support, a sexual stimulation device supported by the support, and a sensor assembly supported by the support. The sensor assembly includes a sensor that is configured to detect an aspect of a sexual activity performed on the sexual stimulation device, and a processor in communication with the sensor for receiving a signal from the sensor, processing the signal, and outputting information relating to the sexual activity.

### FIELD

This document relates to sexual wellness. More specifically, this document relates to systems, methods, and apparatuses for sexual performance sensing.

### BACKGROUND

U.S. Pat. No. 6,368,268 (Sandvick et al.) discloses an interactive virtual sexual stimulation system that has one or more user interfaces. Each user interface generally includes a computer having an input device, video camera, and transmitter. The transmitter is used to interface the computer with one or more sexual stimulation devices, which are also located at the user interface. In accordance with the preferred embodiment, a person at a first user interface controls the stimulation device(s) located at a second user interface. The first and second user interfaces may be connected, for instance, through a web site on the internet. In another embodiment, a person at a user interface may interact with a pre-recorded video feed. The invention is implemented by software that is stored at the computer of the user interface, or at a web site accessed through the internet.

U.S. Pat. No. 10,272,011 (Sloan) discloses an electromechanical device for male sexual stimulation that uses a reciprocal linear motion similar to sexual intercourse, and wherein the penis remains fully inserted during use. The reciprocal linear motion is generated by a small motor which drives a screw and nut mechanism, to which a bracket and gripper is attached. Inserted into the gripper is a flexible sleeve. A penis may be inserted into the device inside the flexible sleeve. The movement of the gripper and sleeve against the penis provides pressure and motion against the penis inside the sleeve in a manner similar to sexual intercourse. The speed, pattern, and location of the motion can be controlled by the user through controls on the outside of the device.

### SUMMARY

The following summary is intended to introduce the reader to various aspects of the detailed description, but not to define or delimit any invention.

Sexual performance sensing apparatuses are disclosed. According to some aspects, a sexual performance sensing apparatus includes a support, a sexual stimulation device supported by the support, and a sensor assembly supported by the support. The sensor assembly includes a sensor that is configured to detect an aspect of a sexual activity performed on the sexual stimulation device, and a processor in communication with the sensor for receiving a signal from the sensor, processing the signal, and outputting information relating to the sexual activity.

In some examples, the sensor assembly further includes an output device in communication with the processor for receiving the information from the processor and communicating the information to a user.

In some examples, the sensor assembly further includes a wireless transmitter in communication with the processor for receiving the information from the processor and transmitting the information to an output device.

In some examples, the sexual stimulation device is a phallus.

In some examples, the support includes a first section to which the sexual stimulation device is removably mounted, and a second section proximate the first section and to which the sensor assembly is mounted. The sexual stimulation device can be removably mounted to the first section by a suction cup. The sensor assembly can be adjustably mounted to the second section.

In some examples, the support is in the form of a backing plate having a first face and a second face that is opposite the first face. The sexual stimulation device and sensor assembly can be supported by the first face. The second face can be positionable against a wall and securable to the wall. The apparatus can further include suction cups for securing the second surface to the wall.

In some examples, the sensor is an infrared sensor, a time of flight sensor, an optical sensor, a triangulation sensor, a capacitive sensor, a radar sensor, a lidar sensor, an accelerometer, a temperature sensor, and/or a pressure sensor.

In some examples, the sensor is configured to detect a distance between a performer and the sensor.

In some examples, the information is at least one of a depth of penetration by the sexual stimulation device in the sexual activity, and a speed of movement in the sexual activity.

In some examples, the apparatus further includes a battery for providing power to the sensor and the processor.

In some examples, the information includes at least one of performance information and coaching information.

According to some aspects, a sexual performance sensing apparatus includes a support and a sensor assembly. The support has a first section to which a sexual stimulation device is mountable, and a second section proximate the first section. The sensor assembly is mountable to the second section, and includes a sensor that is configured to detect aspect of a sexual activity performed on the sexual stimulation device when the sexual stimulation device is mounted to the first section, and a processor in communication with the sensor for receiving a signal from the sensor, processing the signal, and outputting information relating to the sexual activity.

Sexual performance sensing systems are also disclosed. According to some aspects, a sexual performance sensing system includes a sensor configured to detect a distance between a performer and the sensor during a sexual activity, a processor in communication with the sensor for receiving a signal from the sensor, processing the signal, and outputting information that is a function of the distance detected during the sexual activity, and an output device in communication with the processor for receiving the information and communicating the information.

In some examples, the system further includes a sexual stimulation device on which the sexual activity is performable.

In some examples, the system further includes a support, and the sexual stimulation device and the sensor are supported by the support. The output device can be supported by the support, or can be remote from the support and in wireless communication with the processor.

In some examples, the output device includes a visual display. The visual display can be supported by the support.

In some examples, the output device includes a tactile device.

Methods for sexual performance sensing are also disclosed. According to some aspects, a method for sexual performance sensing includes: a. performing a sexual activity on a sexual stimulation device or body part, and b. using a sensor assembly positioned proximate the sexual stimulation device or body part to detect a distance between a participant in the sexual activity and the sensor during the sexual activity, and to output information that is a function of the distance detected during the sexual activity.

In some examples, step a. includes performing oral sex on the sexual stimulation device or body part, and in step b., the information is a depth of penetration of the sexual stimulation device or body part in the oral sex.

In some examples, step a. includes performing oral sex on the sexual stimulation device or body part, and in step b., the information is a speed of movement.

In some examples, in step a., the sexual activity is performed on the sexual stimulation device, and the sexual stimulation device and sensor assembly are mounted to a support.

In some examples, in step a., the sexual activity is performed on a body part, and the sensor assembly is worn by the participant.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example sexual performance sensing system, including a sexual performance sensing apparatus and an auxiliary device;

FIG. 2 is an exploded perspective view of the sexual performance sensing apparatus of FIG. 1;

FIG. 3 is a front view of the sensor assembly of the sexual performance sensing apparatus of FIGS. 1 and 2;

FIG. 4 is a perspective view showing a support of the sexual performance sensing apparatus of FIGS. 1 and 2, and a portion of the sensor assembly of FIG. 3;

FIG. 5A is a schematic side view showing a first step in an example method of sexual performance sensing; and

FIG. 5B is a schematic side view showing a second step in the method of FIG. 5A.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are apparatuses, systems, and methods for sexual performance sensing. The apparatuses may generally include a sexual stimulation device, such as an artificial penis (also referred to herein as a “dildo”), and a sensor assembly. In use, the sexual stimulation device and sensor assembly may be mounted together on a support such as a backing plate, which may in turn be mounted to a fixture such as a wall. A user may then perform a sexual activity using the sexual stimulation device, and the sensor assembly can detect one or more aspects of the sexual activity. For example, the user can perform oral sex on the sexual stimulation device, and the sensor assembly can detect a distance between the sensor and the user (e.g. the user's forehead) over the course of the sexual activity. The sensor assembly can, for example, detect the distance at a frequency of between about 10 and about 100 measurements per second. The sensor assembly can then output information relating to the sexual activity (e.g. information that is a function of the distance detected by the sensor). For example, a duration of the sexual activity, a depth of penetration of the sexual stimulation device during the sexual activity, and/or a speed of the sexual activity can be displayed. The information can be displayed on the apparatus itself, and/or can be transmitted to an auxiliary device (e.g. a smartphone or tablet) for display. Furthermore, a mobile application or website may be provided that can track the user's performance (e.g. long term performance), and can offer coaching. For example, the mobile app may include a dashboard that displays information for various sexual activity sessions over a period of time.

The apparatuses, systems, and methods may have various uses and benefits. For example, the apparatuses, systems, and methods may be used for pleasure and/or amusement, and/or to improve a sexual relationship between a couple, and/or to treat medical conditions (e.g. vaginismus).

As used herein, the term “body part” refers to any human body part used for sexual purposes. For example, the term body part” may include, but is not limited to, a penis, a vagina, a vulva, an anus, a mouth, one or more fingers, one or more breasts, one or more nipples, a scrotum, a tongue, a fist, or one or more toes.

As used herein, the term “sexual stimulation device” refers to any device that may be used for sexual stimulation, with the exclusion of body parts. For example, the term “sexual stimulation device” may include, but is not limited to a dildo, a strap-on dildo, an anal plug, a vibrator, an artificial vagina, or combinations thereof.

As used herein, the term “phallus” refers to a real penis, an artificial penis, or any object that resembles a penis (e.g. a real or artificial finger, an artificial tentacle, or a fantasy phallus).

As used herein, the term “sexual activity” refers to any activity carried out by a human on a sexual stimulation device or body part (whether their own or someone else's) for sexual purposes. The term “sexual activity” may include, but is not limited to, intercourse, oral sex, anal sex, penetration (e.g. anal, oral, or vaginal penetration) with a sexual stimulation device, and/or masturbation.

As used herein, the term “user” may refer to a participant in a sexual activity (i.e. a performer of a sexual activity or a recipient of a sexual activity), and/or a bystander to a sexual activity. For example, the term “user” may refer to a person performing oral sex, a person receiving oral sex, a person being penetrated, a person penetrating another person, and/or two or more people engaged in intercourse. For further example, the term “user” may refer to a person that is observing or tracking the sexual activity of another person or people (e.g. a dominant observing or tracking the activity of a submissive, or a person observing or tracking the activity of a partner or patient).

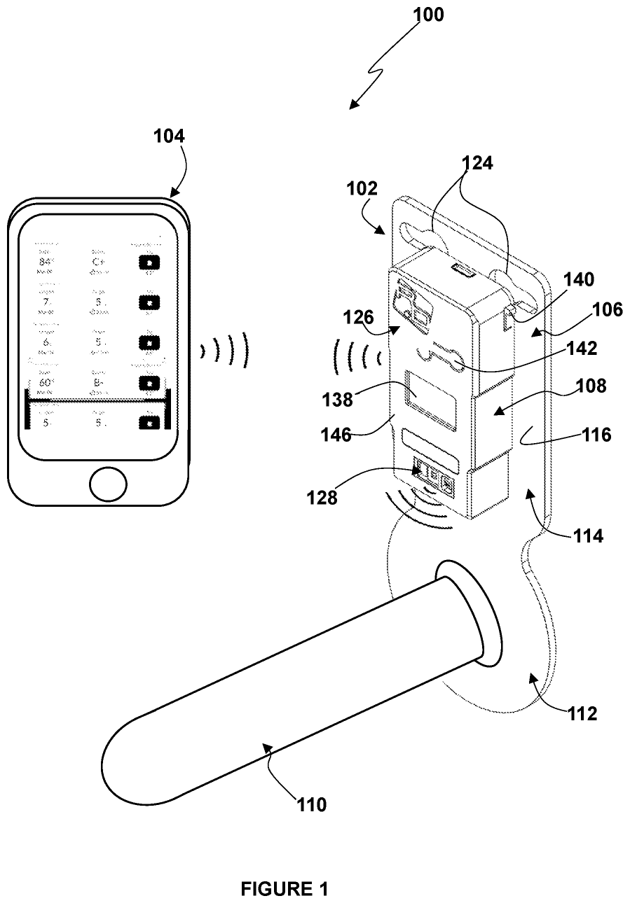

Referring now to FIG. 1, an example sexual performance sensing system 100 is shown. The system 100 generally includes a sexual performance sensing apparatus 102, and an auxiliary device 104 that is in communication with the apparatus 102. As will be described in greater detail below, the apparatus 102 can generally be used to obtain information relating to a sexual activity, and the auxiliary device 104 can be used to communicate the information to a user (e.g. via a display) and/or to store the information. In the example shown, the auxiliary device 104 is in the form of a smart phone. In alternative examples, the auxiliary device 104 may be in another form, such as a tablet, smart watch, a laptop computer, a virtual reality headset, or a desktop computer. In further alternative examples, the auxiliary device 104 may be omitted, and the information may be displayed and/or stored by the apparatus 102 itself, and may optionally be retrieved and/or analyzed.

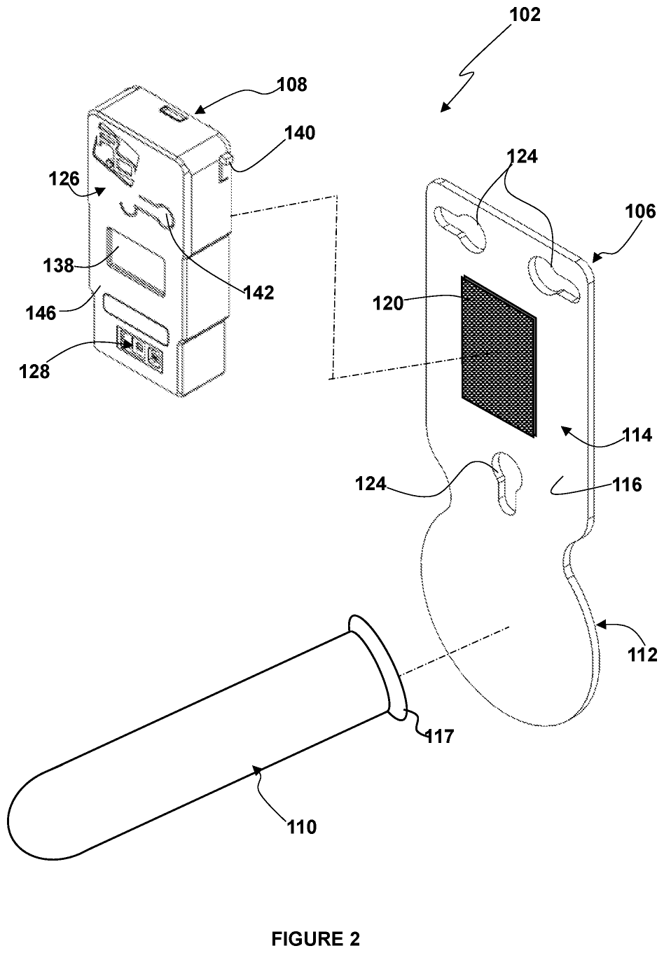

Referring now to FIG. 2, the apparatus 102 is shown in greater detail. In the example shown, the apparatus 102 includes a support 106, a sensor assembly 108, and a sexual stimulation device 110 in the form of a dildo. As noted above, in other examples, various other sexual stimulation devices may be used in the apparatus.

Referring still to FIG. 2, in use, the support 106 serves to hold the sensor assembly 108 and the sexual stimulation device 110 in place. In the example shown, the support 106 is in the form of a backing plate and includes a first section 112 that supports the sexual stimulation device 110, and a second section 114 that supports the sensor assembly 108. The second section 114 is proximate the first section 112 (i.e. in the example shown they are side-by-side), so that in use, the sensor assembly 108 is sufficiently close to the sexual stimulation device 110 to detect sexual activity carried out using the sexual stimulation device 110.

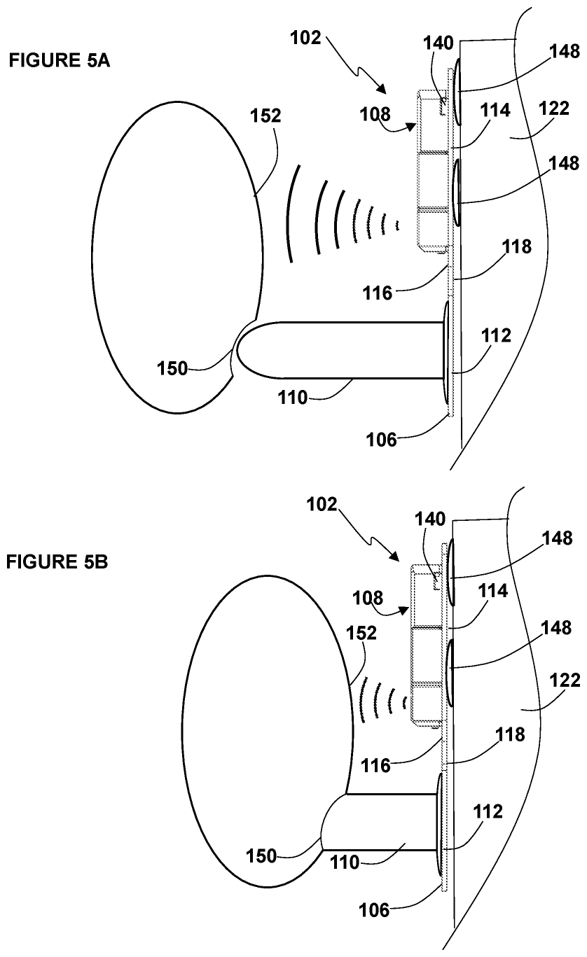

Referring still to FIG. 2, in the example shown, the support 106 includes a first face 116 (i.e. a front face) and a second face 118 (i.e. a back face, shown in FIGS. 5A and 5B) that is opposite the first face. Both the sexual stimulation device 110 and the sensor assembly 108 are supported by the first face 116. More specifically, in the example shown, the sexual stimulation device 110 is removably mountable to the first face 116 via a suction cup 117. This configuration allows for a user to use various sexual stimulation devices in the apparatus. That is, the use of releasable fasteners such as suction cups can allow for a user to, for example, select a dildo or an anal plug to use in the apparatus, or select the size of dildo used in the apparatus (e.g. the user may initially use a relatively small dildo, and may then upgrade to a larger dildo). Furthermore, the sensor assembly 108 is removably and adjustably mountable to the first face 116 using a hook-and-loop fastener (e.g. Velcro®), where a first sheet 120—i.e. either a hook sheet or a loop sheet—is fixed to the first face 116, and a second sheet (not shown)—i.e. either a hook sheet or a loop sheet—is fixed to a back side of the sensor assembly 108. This configuration allows for the user to adjust the position of the sensor assembly 108. For example, depending on various factors, such as the use of the apparatus 102 and/or the anatomy of the user, the position of the sensor assembly 108 with respect to the sexual stimulation device 110 may benefit from adjustment in order to obtain accurate readings. The use of an adjustable faster such as a hook-and-loop fastener can facilitate this adjustment.

In alternative examples, other fasteners may be used. For example, another type of fastener may be used to removably mount the sexual stimulation device 110 to the support 106, or the sexual stimulation device 110 may be permanently fixed to the support 106. Likewise, another type of adjustable or non-adjustable fastener may be used to removably mount the sensor assembly 108 to the support 106, or the sensor assembly 108 may be permanently fixed to the support 106.

The second face 118 of the support 106 is positionable against and securable to a fixture such as a wall 122 (shown in FIGS. 5A and 5B). For example, suction cups 148 (shown in FIGS. 5A and 5B) may be secured in mounting holes 124 in the support 106, to allow for the second face 118 to be removably secured to a wall. In alternative examples, the second face 118 may be securable to a wall 122 in another fashion, for example using bolts or screws. In further examples, the apparatus 102 may be used in a free-standing configuration (e.g. may be handheld or placed on a floor), or may be mounted to another apparatus (e.g. an actuated mount that moves relative to the user).

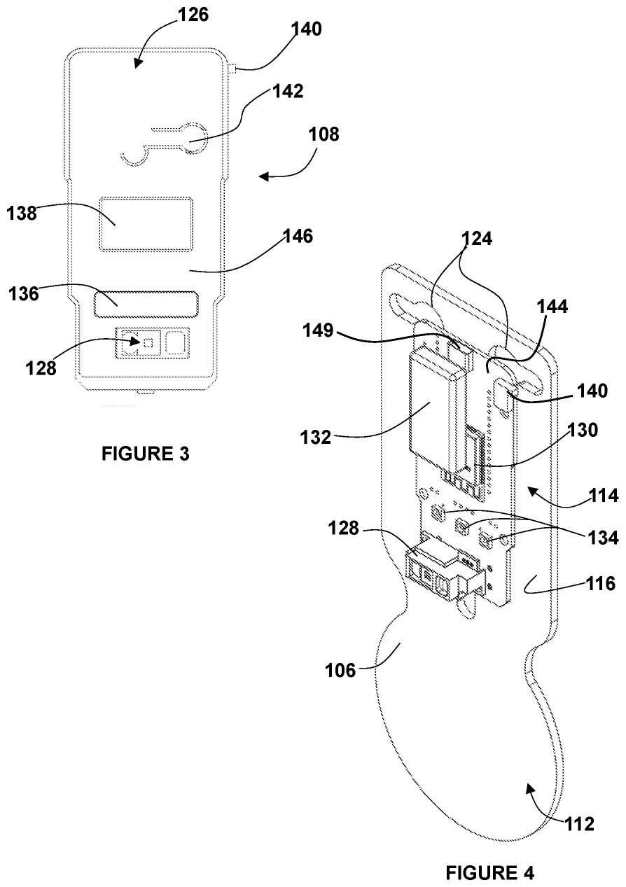

Referring now to FIGS. 3 and 4, the sensor assembly 108 is shown in greater detail. In the example shown, the sensor assembly 108 generally includes a casing 126, a sensor 128, a processor 130, a battery 132, a first output device in the form of a colored light indicator that includes a set of light emitting diodes (LEDs) 134 (e.g. RGB multicolor LEDs and/or individually addressable LEDs) and a screen 136, and a second output device in the form of a liquid crystal display (LCD) screen 138, an on/off switch 140, and a control button 142.

The casing 126 includes a base plate 144 (shown in FIG. 4) to which various components of the sensor assembly (e.g. the battery 132 and the processor 130) are mounted, and a cover 146 (shown in FIG. 3) that encloses the components of the sensor assembly 108 and to which various other components of the sensor assembly (e.g. the screen 136 and the LCD screen 138) are mounted.

Referring still to FIGS. 3 and 4, the sensor 128 is configured to detect an aspect of a sexual activity performed on the sexual stimulation device 110. In the example shown, the sensor 128 is in the form of an infrared triangulation sensor that is directed outwardly through a window in the cover 146. The sensor 128 is configured to detect a distance between the sensor 128 and an object in front of the sensor 128. For example, in the case of a user performing oral sex on a dildo secured to the support 106, the sensor 128 can detect a distance between the user's forehead and the sensor 128. The sensor 128 can be configured to take measurements at a frequency of, for example between 10 Hz and 100 Hz (e.g. about 60 Hz).

In alternative examples, the apparatus 102 can include another type of sensor, which can be configured to detect another aspect of a sexual activity. For example the sensor can be another type of infrared sensor, a time of flight sensor, an optical sensor, another type of triangulation sensor, a capacitive sensor, a radar sensor, a lidar sensor, an accelerometer, a temperature sensor, or a pressure sensor. The sensor can be a digital sensor or an analog sensor. In further alternative examples, the apparatus may include more than one sensor or more than one type of sensor, or more than one sensor assembly.

Referring to FIG. 4, the processor 130 is in communication with the sensor 128 (e.g. wired or wireless communication), and can receive a signal from the sensor 128, process the signal, and output the information relating to the sexual activity. More specifically, in the example shown, in which the sensor 128 is a distance sensor, the processor 130 can process the signal from the sensor 128 and then output information that is a function of the sensed distance, such as the depth of penetration of the dildo during the sexual activity, and/or the speed of movement in the sexual activity. Additional information, such as the duration of the sexual activity, can also be output by the processor 130. The processor 130 can output the information by communicating the information to the LCD screen 138 for display to a user, to the colored light indicator for display to a user, and/or to an output device that is remote from the apparatus (e.g. to the auxiliary device 104) for display to the user. In examples where information is communicated to the LCD screen 138 and/or the colored light indicator, this can optionally be achieved via wired communication between the processor 130 and the LCD screen 138 and between the processor 130 and the LEDs 134. In examples where information is communicated to the auxiliary device 104, this can be achieved via a wireless transmitter included in the processor 130 of the sensor assembly 108.

The LCD screen 138 and colored light indicator can display various information to the user, such as coaching information and/or tracking information. Tracking information can include, for example, a depth of penetration of the sexual stimulation device 110 during the sexual activity, and/or a speed of movement during the sexual activity, and/or a time for which the apparatus 102 has been used, and/or a number of repetitions performed, and/or pace of the sexual activity, and/or quality of the sexual activity (e.g. based on whether the pattern of movement in the sexual activity matched a pre-set pattern) and/or whether depth was maintained. Coaching information can include, for example, tips and/or guidance relating to performance improvement. For example, the LCD screen 138 can display a time remaining or time spent in a training session, and/or a depth of penetration of the sexual stimulation apparatus 110, and/or a number of repetitions performed or remaining, while the colored light indicator can, for example, turn yellow to coach the user into slowing down, or turn green to coach the user to speed up, or turn red to coach the user to stop.

The auxiliary device 104 can also display various information to the user. For example a mobile application or website may be provided that can track long term performance. For example, a dashboard may be provided that can display tables or graphs of a user's performance (e.g. showing depth or endurance over various training dates). Furthermore, the mobile application or website can be used to develop a training schedule, or to assign tasks (e.g. a dominant may assign training tasks to a submissive using the mobile application or website, or a therapist or doctor may assign tasks to a patient).

In alternative examples, the sensor assembly can include a physical output port (e.g. a USB port or audio jack), for wired connection of the sensor assembly to one or more auxiliary devices.

In the example shown, information is communicated to the user(s) via output devices in the form of a plurality of visual displays. In alternative examples, information may be communicated in another manner, and alternative output devices may used. For example, the apparatus or the auxiliary device may incorporate a speaker for communicating information to the user(s), optionally in real time. For further example, information may be communicated to the user in a tactile fashion. For example, information may be communicated via vibrations of the sexual stimulation device, or by tactile stimulation sent by another device (e.g. a sex machine or shock device). For further example, another type of visual display (e.g. an OLED screen) may be used.

Referring still to FIG. 4, in the example shown, the battery 132 is a rechargeable battery that can be charged via a port 149 (e.g. a micro-USB or USB-C port) in the casing 126. In alternative examples, the sensor assembly 108 can include one or more single-use batteries, and the sensor assembly 108 can be configured for regular replacement of the batteries. In further alternative examples, the apparatus 102 can be configured to plug directly into a wall outlet, and the battery 132 can be omitted.

Referring now to FIGS. 5A and 5B, a method of sexual performance sensing will be described. The method will be described with regards to the system 100 and apparatus 102 of FIGS. 1 to 4; however, the method can be performed with other systems and apparatuses, and the system 100 and apparatus 102 can be used according to other methods. As will be described in greater detail below, the method can generally include performing a sexual activity on a sexual stimulation device or body part of a recipient, and using a sensor assembly positioned proximate the sexual stimulation device or body part to detect a distance between a performer of the sexual activity and the sensor during the sexual activity, and to output information that is a function of the distance detected during the sexual activity.

Referring first to FIG. 5A, the apparatus 102 can optionally be secured to wall 122 using suction cups 148 on the second face 118 of the support 106. The sensor assembly 108 and sexual stimulation device 110 can be secured to the first face 116 of the support 106, with the sexual stimulation device 110 secured to the first section 112 and the sensor assembly 108 secured to the second section 114. The sensor assembly 108 can then be turned on using the on/off switch 140.

In some examples, on startup, a setup method can first be performed in order to input the type of sexual stimulation device and type of sexual activity being performed. For example, the LCD screen 138 (not visible in FIGS. 5A and 5B) can display a list of sexual stimulation devices and a list of sexual activities. Using the control button 142 (not visible in FIGS. 5A and 5B), the user can select the sexual stimulation device that they plan to use (e.g. a dildo), the sexual activity that they plan to perform (e.g. oral sex), and/or the size of the dildo. Alternatively, this information can be input into the auxiliary device 104 (not shown in FIGS. 5A and 5B).

Then, a calibration method can then be performed in order to assess the user's initial capabilities. For example, the LCD screen 138 can display instructions telling the user to place only the tip of the sexual stimulation device 110 in the user's mouth 150, as shown in FIG. 5A. When the user does this, the sensor 128 (not visible in FIGS. 5A and 5B) can detect the distance to the user's forehead 152 (or to another surface that is in front of the sensor 128, such as the user's nose), and send a signal to the processor 130 (not visible in FIGS. 5A and 5B). The LCD screen 138 can then display instructions telling the user to place the sexual stimulation device 110 as far into their mouth 150 as possible, as shown in FIG. 5B. When the user does this, the sensor 128 can detect the distance to the user's forehead 152, and send a signal to the processor 130. The processor 130 can then determine the length of sexual stimulation device 110 initially placed in the user's mouth 150, and/or determine the user's initial skill level or ability.

After the calibration method is complete, the user can perform the sexual activity on the sexual stimulation device 110, optionally with the coaching of the system 110. For example, instructions can be displayed on the LCD screen 138, a colored light (e.g. green) can be displayed by the colored light indicator, or auditory instructions can be played by the auxiliary device 104. Over the course of the sexual activity, the sensor assembly 108 can detect the distance between the user's forehead 152 and the sensor 128, and can output information that is a function of the distance (e.g. information relating to depth of penetration and/or speed of movement of the performer). For example, in real time, the LCD display can display text advising the user to slow down, or to attempt to place the sexual stimulation device 110 deeper into their mouth 150. Furthermore, the colored light indicator can display, for example, a red light to indicate to the user to stop, or a yellow light to indicate to the user to slow down, or a green light to indicate to the user to keep going. Furthermore, over the course of the sexual activity or afterwards, the sensor assembly 108 can transmit information to the auxiliary device 104, so that long term data can be tracked and displayed using the dashboard.

Optionally the system 100 can guide the user through a set of predetermined exercises (e.g. a certain number of repetitions of a certain depth of penetration), such as a set of exercises assigned by a partner, a professional such as a therapist, doctor, or trainer, or by an online community.

In another example, rather than using the sensor assembly 108 with a sexual stimulation device, the sensor assembly can be used with a body part of a participant in a sexual activity. For example, the sensor assembly 108 can be worn by attaching the sensor assembly 108 to a belt, and can be used during the course of oral sex performed on the body part of recipient.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A sexual performance sensing apparatus, comprising:
a support;
a sexual stimulation device supported by the support;
a sensor assembly supported by the support, the sensor assembly comprising a sensor that is configured to detect an aspect of a sexual activity performed on the sexual stimulation device, and a processor in communication with the sensor for receiving a signal from the sensor, processing the signal, and outputting information relating to the sexual activity.

2. The sexual performance sensing apparatus of claim 1, wherein the sensor assembly further comprises an output device in communication with the processor for receiving the information from the processor and communicating the information.

3. The sexual performance sensing apparatus of claim 1, wherein the sensor assembly further comprises a wireless transmitter in communication with the processor for receiving the information from the processor and transmitting the information to an output device.

4. The sexual performance sensing apparatus of claim 1, wherein the support comprises a first section to which the sexual stimulation device is removably mounted, and a second section proximate the first section and to which the sensor assembly is mounted.

5. The sexual performance sensing apparatus of claim 4, wherein the sensor assembly is adjustably mounted to the second section.

6. The sexual performance sensing apparatus of claim 1, wherein the support is in the form of a backing plate having a first face and a second face that is opposite the first face, wherein the sexual stimulation device and sensor assembly are supported by the first face.

7. The sexual performance sensing apparatus of claim 6, wherein the second surface is positionable against a wall and securable to the wall.

8. The sexual performance sensing apparatus of claim 1, wherein the sensor is configured to detect a distance between a performer and the sensor.

9. The sexual performance sensing apparatus of claim 1, wherein the information is at least one of a depth of penetration by the sexual stimulation device in the sexual activity and a speed of movement in the sexual activity.

10. A sexual performance sensing system, comprising:
a sensor configured to detect a distance between a performer and the sensor during a sexual activity;
a processor in communication with the sensor for receiving a signal from the sensor, processing the signal, and outputting information that is a function of the distance detected during the sexual activity; and
an output device in communication with the processor for receiving the information and communicating the information.

11. The system of claim 10, further comprising a sexual stimulation device on which the sexual activity is performable.

12. The system of claim 11, further comprising a support, wherein the sexual stimulation device and the sensor are supported by the support.

13. The system of claim 10, wherein the output device is supported by the support.

14. The system of claim 10, wherein the output device is remote from the support and is in wireless communication with the processor.

15. The system of claim 10, wherein the output device comprises a visual display.

16. The system of claim 15, wherein the visual display is supported by the support.

17. A method for sexual performance sensing, comprising:
a. performing a sexual activity on a sexual stimulation device or body part; and
b. using a sensor assembly positioned proximate the sexual stimulation device or body part to detect a distance between a participant in the sexual activity and the sensor during the sexual activity, and to output information that is a function of the distance detected during the sexual activity.

18. The method of claim 17, wherein:
step a. comprises performing oral sex on the sexual stimulation device or body part; and
in step b., the information is a depth of penetration of the sexual stimulation device or body part in the oral sex, and/or a speed of movement.

19. The method of claim 17, wherein in step a., the sexual activity is performed on the sexual stimulation device, and wherein the sexual stimulation device and sensory assembly are mounted to a support.

20. The method of claim 17, wherein in step a., the sexual activity is performed on a body part, and wherein the sensor assembly is worn by a participant.
